(19) United States
(12) Patent Application Publication
(10) Pub. No.: US 2011/0083983 A1
(43) Pub. Date: Apr. 14, 2011

(54) SURGICAL FIELD ORGANIZER

(75) Inventors: Michael W. WALTERS, Marshall, MI (US); Jeffrey L. Cornell, Coldwater, MI (US); David R. Mead, Marshall, MI (US); Michael S. Smith, Hastings, MI (US)

(73) Assignee: Progressive Dynamics, Inc., Marshall, MI (US)

(21) Appl. No.: 12/690,261

(22) Filed: Jan. 20, 2010

Related U.S. Application Data
(63) Continuation-in-part of application No. 12/578,800, filed on Oct. 14, 2009.

(51) Int. Cl. A61B 19/02

(52) U.S. Cl. 206/370; 206/438

(57) ABSTRACT

An instrument holder for use alone or in combination with a hose/cable organizer. The holder can be made in several forms; the preferred embodiment being an elongate plastic body with fold-up wing structures carrying opposed sets of instrument receivers such as spring fingers, loops, cups and combinations thereof. The holder may be used alone or in combination with a hose/cable organizer.

### RELATED APPLICATION

This application is a continuation-in-part of U.S. application Ser. No. 12/578,800 filed under attorney docket no. PDY-118-A on Oct. 14, 2009, currently pending. The content of the U.S. patent application Ser. No. 12/578,800 is incorporated herein by reference.

### FIELD OF THE INVENTION

This invention relates to equipment organizers and instrument holders for use in a surgical field and more particularly to devices which may be adhered such as by tape to a surgical field drape to provide either an organizer for hoses, fiber optics, cables and other flexible connectors, or a convenient and secure resting place for one or more articles such as surgical instruments, or a combination of the two.

### BACKGROUND OF THE INVENTION

An OR surgical site for even relatively simple procedures can be characterized by a large number of hoses, tubes, wires, cables and the like which extend into the surgical field from peripheral sites to supply power, fluids, gasses, suction and data connections to various instruments such as probes, endoscopes, aspirators, Bovies, drills and other handheld instruments. A typical OR situation often results in hoses, cables, wires, tubes and the like lying on and around the patient's body, primarily on top of the surgical drape and within the sterile field. Should one or more of these instruments fall outside of the sterile field, it must be replaced before the procedure can be started or resumed. Resting places for instruments can be provided by shelves, trays and the like and often involve handing instruments from person to person during a procedure.

### SUMMARY OF THE INVENTION

The present invention, according to one aspect, provides a simple, economical device susceptible of sterilization and/or sterile packaging which is useful in both organizing and providing a secure resting place for elongate, handheld articles such as surgical instruments of the type described above. The invention, therefore, not only contributes to orderliness in various scenarios, but can also facilitate procedures and makes them more efficient.

According to another aspect of the invention, a device is provided for the organization of the hoses, cables, fiber optics, and wires associated with various procedures including surgical procedures. The two aspects; i.e., the instrument holder and the cable and/or hose organizer, can work in combination or alone and both are susceptible of being secured, such as by two-sided tape, to a surgical drape or other surface and/or to each other.

In general, a cable and/or hose organizer comprises a hollow body which defines one or more channels within which a plurality of tubes, hoses, cables and the like can be gathered and held at a central site. The organizer can, by way of example, have a hinged or clip-on top for ease of installing the hoses, cables, etc. into the organizer and may be configured in such a way as to receive or incorporate an instrument holder therewith.

In the preferred form, the instrument holder comprises a flat plastic body with multiple instrument receivers upstanding therefrom in the final configuration. The receivers are made up of opposed, flexible clips of various sizes and/or a clip in opposed, aligned relationship with an open loop or a closed-end cup. The entire device may be made of plastic which can be sterilized or resterilized and packaged in a flexible bag or pouch. Both devices are light in weight and inexpensive enough to be disposable. The holder and organizer preferably have flat bottoms provided with two-sided sterile tape or other fastener system which can be used to secure the body of the device to each other and/or to a surgical drape. The body can take many shapes, several of which are illustrated herein. In the preferred form, the holder is injection molded fairly flat with edge wings which are folded up and latched in place to provide the instrument receivers.

Other applications of the present invention will become apparent to those skilled in the art when the following description of the best mode contemplated for practicing the invention is read in conjunction with the accompanying drawings.

### BRIEF DESCRIPTION OF THE DRAWINGS

The description herein makes reference to the accompanying drawings wherein like reference numerals refer to like parts throughout the several views and wherein:

FIG. 1 is a perspective view of a first embodiment of the organizer aspect of the invention with an attached cover opened to provide access to a T-shaped channel body having two inlets and an outlet;

FIG. 2 is a perspective view of an embodiment of the organizer integrated with an instrument holder having four push-in clamp-type instrument receivers mounted in side-by-side fashion to the top of the cover;

FIG. 3 is a perspective view of a second embodiment wherein the cover, rather than being hinged to the channel body as shown in FIG. 2, is completely detachable therefrom;

FIG. 4 is a perspective view of a surgical site showing an embodiment of the invention secured to the surgical drape and holding a number of different surgical instruments;

FIG. 5 is a perspective view of another embodiment of the holder invention in a pre-use configuration;

FIG. 6 is a perspective view of the embodiment of FIG. 5 after it is reconfigured for use and attached by tape to the top of a channel-forming body;

FIG. 7 is a perspective view of another embodiment of the organizer invention;

FIG. 8 is a perspective view of still another embodiment of the organizer invention with still another body shape;

FIG. 9 is a perspective view of another embodiment of the organizer invention;

FIG. 10 is a perspective view of another embodiment of the holder invention in a pre-use configuration; and

FIG. 11 shows the FIG. 10 embodiment reconfigured for use; and

FIG. 12 shows the FIG. 10 embodiment in a sterile package.

### DETAILED DESCRIPTION OF THE ILLUSTRATIVE EMBODIMENTS

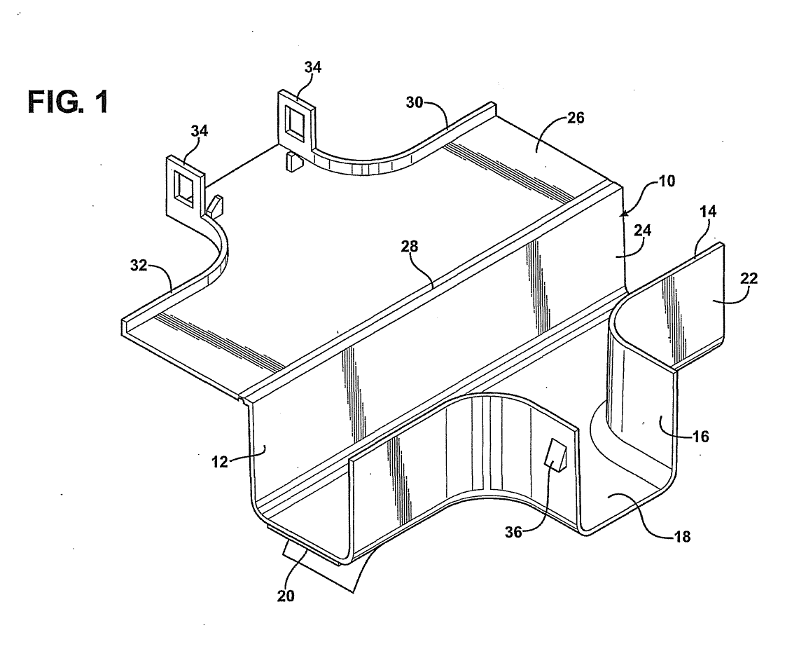

Referring to FIG. 1, there is shown an organizer in the form of a T-shaped plastic body 10 having opposed, in-line inlets 12, 14 and an orthogonal outlet 16. The body 10 is about 5 inches in width and comprises a floor 18 and integral upstanding sidewalls 22, 24. A cover 26 is integrally connected to the body 10 by means of a living hinge 28. The cover exhibits edge flanges 30, 32 terminating in lock frames 34 which receive cam-shaped tabs 36 on the sidewalls 22; only one of the lock tabs 36 is visible in FIG. 1. The cover 26 can be rotated around the axis of the hinge 28 into a position where it closes the T-shaped channel formed in the organizer body 10. The drawing is to scale.

The organizer 10 is preferably made of a suitable plastic such as polypropylene or polyethylene which can be injection molded and which can be autoclaved or otherwise sterilized for use in a surgical field. It can be made of many other materials including metals such as stainless steel, organic materials, ceramics and others. In practice, it may be sterilized by the manufacturer and shipped in a hermetically sealed, sterile plastic package such as a bag 61 as shown in FIG. 3. Two-sided tape 20 is or can be attached to the bottom surface of the organizer to allow it to be secured to a support surface.

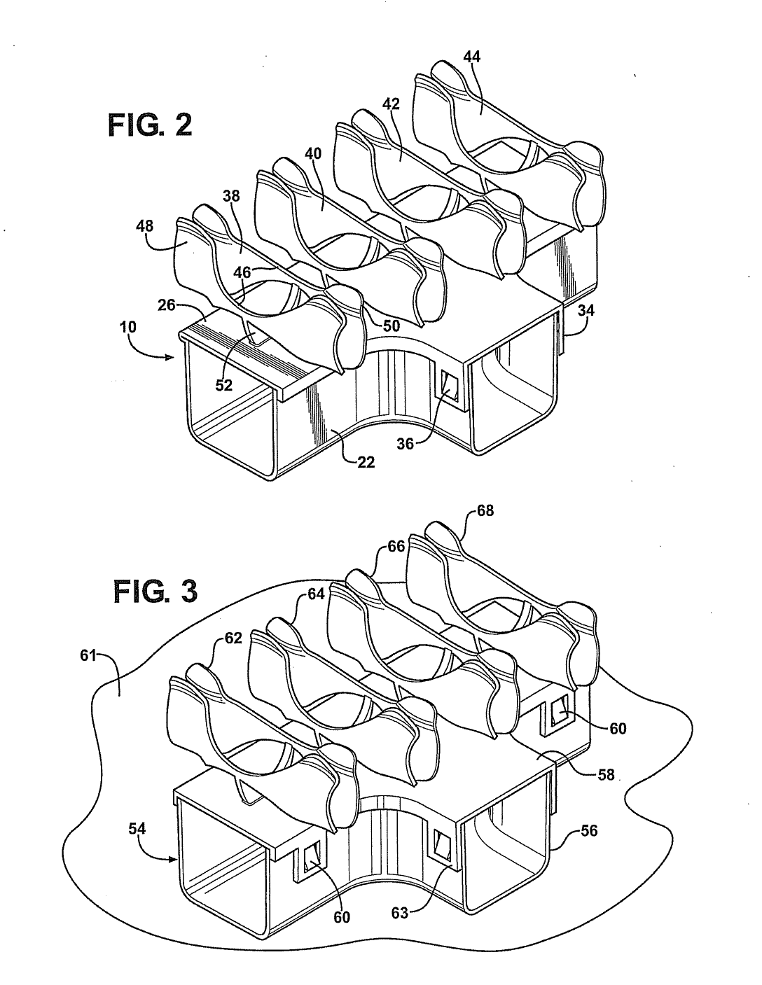

Referring to FIG. 2, the organizer 10 is shown with the cover 26 in a closed position on the lower body and with the latch brackets 34 snapped around the tabs 36.

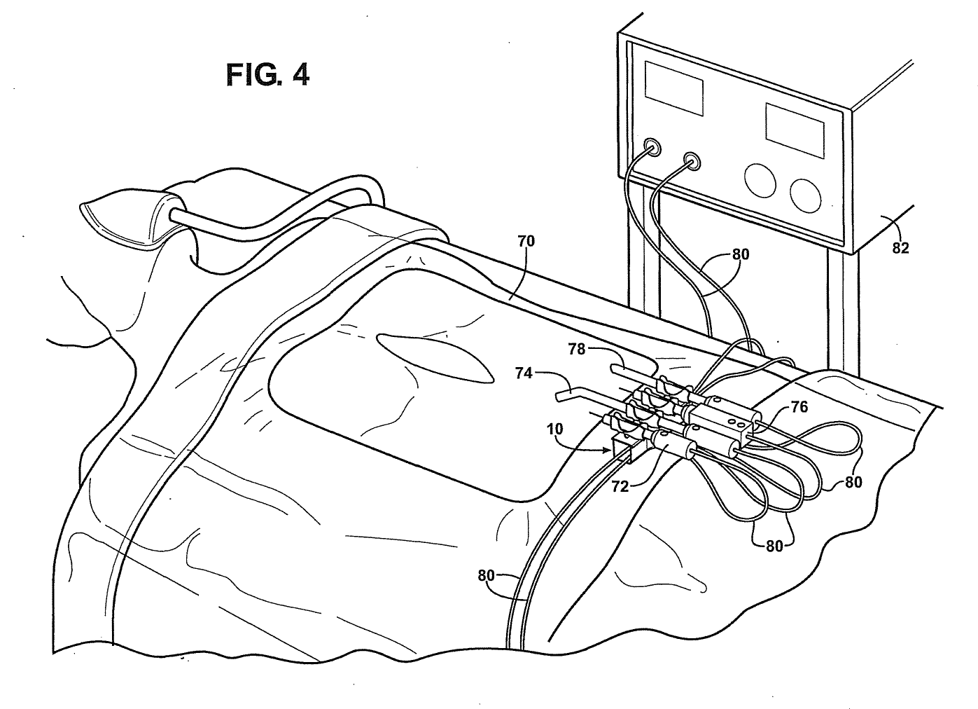

Mounted to the top surface of the cover 26 are flexible push-in type handheld instrument receivers 38, 40, 42 and 44, each of which is made up of two opposed semi-cylindrical clips 46 having raised and spaced apart outwardly flaring labial tabs 48, 50 so that a handheld elongate instrument, such as one of the instruments 72, 74, 76, 78 shown in FIG. 4 can be pushed between the sidewalls 46 and snapped into the receiver 38, 40, 42 or 44 where it is held firmly in position ready for access when needed. The sidewalls 46 are contoured or relieved between the end tabs 48, 50 to permit the barrel of the instrument to be grasped between the fingers and lifted out of the holder 38, 40, 42 or 44 with only appropriate resistance. Each holder side clip 46 has a base 52 used to attach each clip to the top surface of cover 26. The bases preferably snap into holes in the cover in a known manner.

FIG. 3 shows an alternative embodiment of the invention in the form of an organizer 54 having a T-shaped plastic body 56 and a separate T-shaped cover 58 which is secured to the body 56 by means of lock tabs 60 and square frames 63 which receive the tabs 60. There are preferably six such locking arrangements on the organizer 54. Instrument receivers 62, 64, 66 and 68 are arranged in side-by-side, parallel spaced apart relationship and integral with the cover 58 in the same manner as the holders 38, 40, 42 and 44 in the embodiment of FIG. 2. The configuration of the organizer 54 is essentially the same as the configuration of the organizer 10 except for the manner in which the covers are attached to the channel bodies. FIG. 3 also illustrates the organizer 54 packaged in a sterile plastic package, here in the form of a bag 61 which is hermetically sealed at the point of manufacture and opened at the surgical site.

FIG. 4 shows how the organizer 10 may be used in a surgical field. The two-sided tape 20 is used to bond the organizer 10 to the surgical drape 70. Of course, hook and loop fasteners, buttons and other fasteners can be substituted for tape 20. Handheld instruments 72, 74, 76 and 78 are shown latched into the receivers 38, 40, 42 and 44. The elongate flexible appendages; e.g., tubes, hoses, cables, wires, data lines and the like, are looped through the T-shaped channel provided by the organizer 10 so that the elongate flexible connectors 80 extend in an organized and secure fashion off to control centers 82 which may be electronic devices, oxygen supplies, computers, displays, power supplies, pumps, fluid bags and other devices typically found in the surgical environment of an OR or clinic.

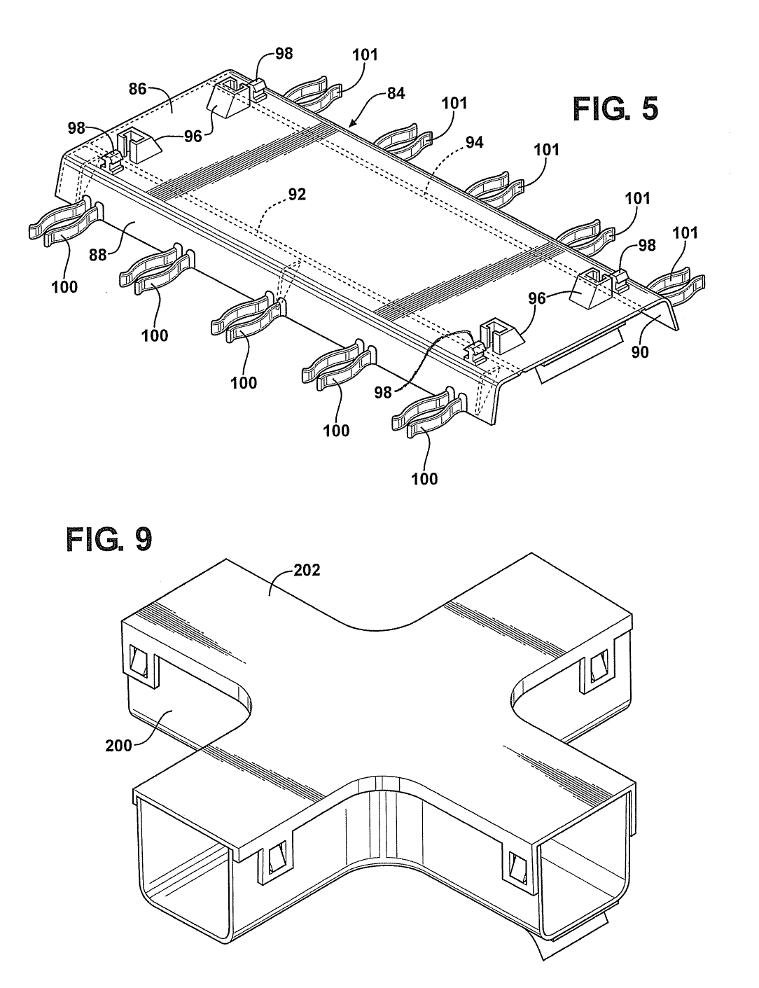

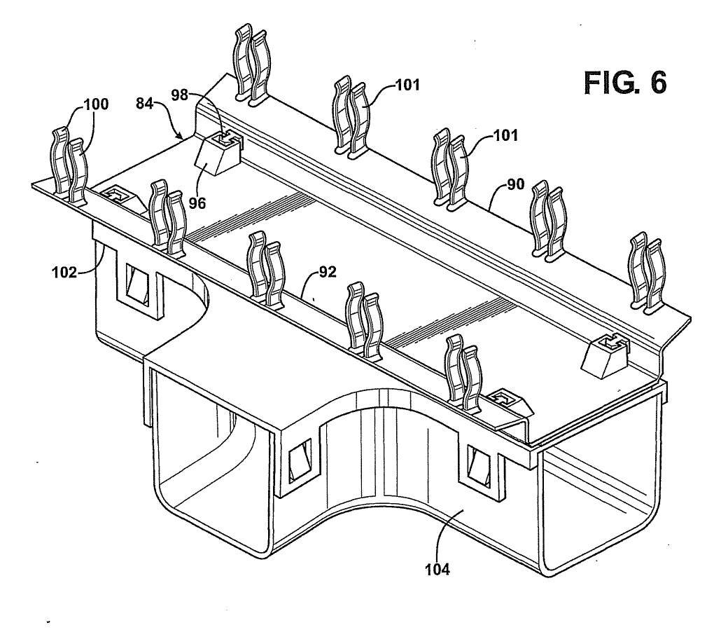

Referring now to FIG. 5, another embodiment of the invention is shown in the form of a molded plastic surgical instrument holder 84 comprising a generally rectangular plastic planar body 86 having laterally opposite, integral, longitudinally extending wing structures 88, 90, each of which is L-shaped in cross-section. The wing members 88, 90 are joined to the body 86 by integral living hinges 92, 94 which run longitudinally and parallel to one another. It is to be understood that FIG. 5 shows the instrument receiver 84 in the configuration corresponding to the design of the mold from which the article is injection molded. It is also packaged in this configuration and is reconfigured for use as hereinafter described with reference to FIG. 6.

Continuing with the description of the instrument receiver 84, female latch members 96 are molded integrally with the rectangular body 86 inside of the hinges 92, 94 and near the longitudinally opposite ends of the body 86. Male fastener members 98 are molded into the wing members 88, 90 on the outside of the living hinges 92, 94 and immediately adjacent the female fasteners 96 whereby the wing members 88, 90 may be rotated upwardly about the hinges 92, 94 until the male latch members 98 snap into the female latch members 96 to hold wing structures in the rotated and raised position shown in FIG. 6.

Wing member 88 carries five sets of spring clips 100 in uniformly spaced relationship. Similarly, wing member 90 carries five sets of opposed spring clips 101. The clips 100 are directly laterally across from the clips 101 such that when the wing members 88, 90 are raised to the position shown in FIG. 6, the clips 100, 101 form aligned pairs approximately three to four inches apart such that a surgical instrument may be readily nested into and between the aligned pairs of opposed spring clips 100, 101 and held in position ready for use. Again, because the spring clips 100, 101 are several inches apart, the entire center body of the instrument is exposed so that it may be easily grasped by the fingers of the surgeon in use. The device of FIGS. 5 and 6 has an advantage over the FIGS. 1-3 embodiments in that it comprises fewer parts and requires less hand assembly.

FIG. 6 shows how the instrument receiver body 84 can be attached to the cover 102 of a T-shaped flexible connector organizer 104 having generally the configuration of the device shown in FIG. 1. The bottom of the body 86 is flat and can be secured by two-sided tape to the top of the cover 102 or, in the alternative, attached to the surgical drape near the connector organizer 104 such that the two may operated in a concerted fashion despite the fact that they are not directly interconnected.

Referring to FIG. 7, there is shown a further embodiment of the invention in the form of a connector organizer 106 which is H-shaped so as to have two inlets 112, 114 and two outlets 116, 118 formed by the combination of the molded plastic body 108 and the hinged clip-on cover 110. The surgical instrument holder may be used in concert with the organizer 106 in the fashion described above; i.e., it may be attached to the cover 110 or integrated into it in the fashion shown in FIG. 2.

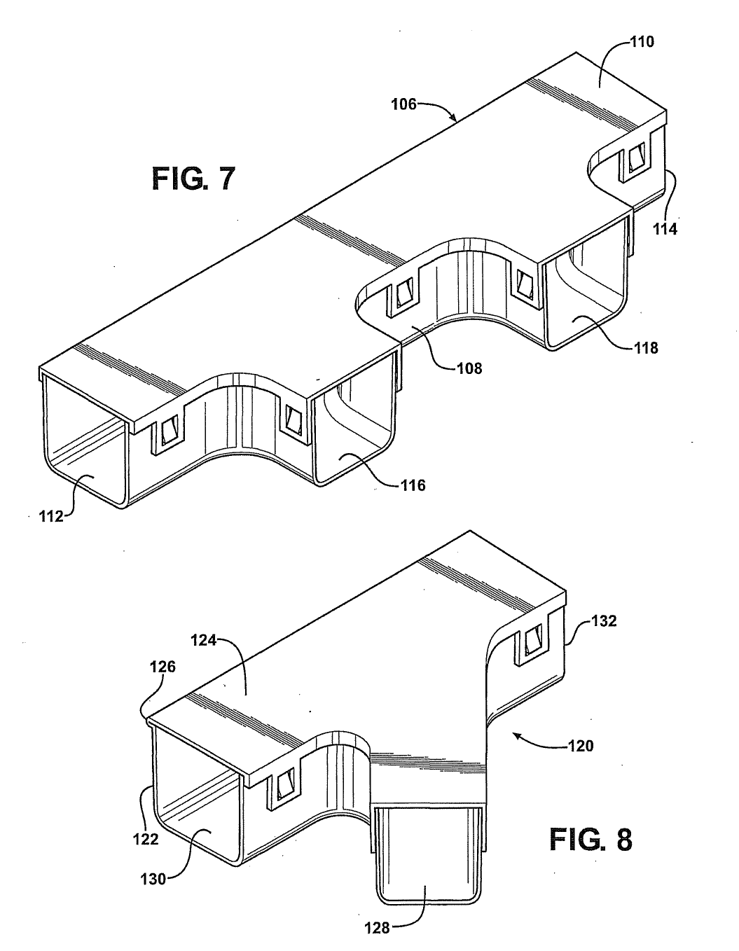

FIG. 8 shows a still further embodiment of the invention, this time in the form of an essentially Y-shaped connector organizer 120 having inlets 128, 130 communicating with an outlet 132. The body 122 has a cover 124 which is connected integrally to it by means of a living hinge 126. Again, the cover 124 may be formed separately and attached to the body 122 by snaps and may operate in concert with a surgical instrument receiver of the type shown in either FIG. 2 or FIG. 6. All of the devices shown in FIGS. 5 through 8 may be sterilized and packaged in a sterilized package, such as a plastic bag, shipped from the factory in ready-to-use in the operating room.

FIG. 9 shows still another form for the organizer, here in a cruciform shape with a body 200 and a snap on top 202.

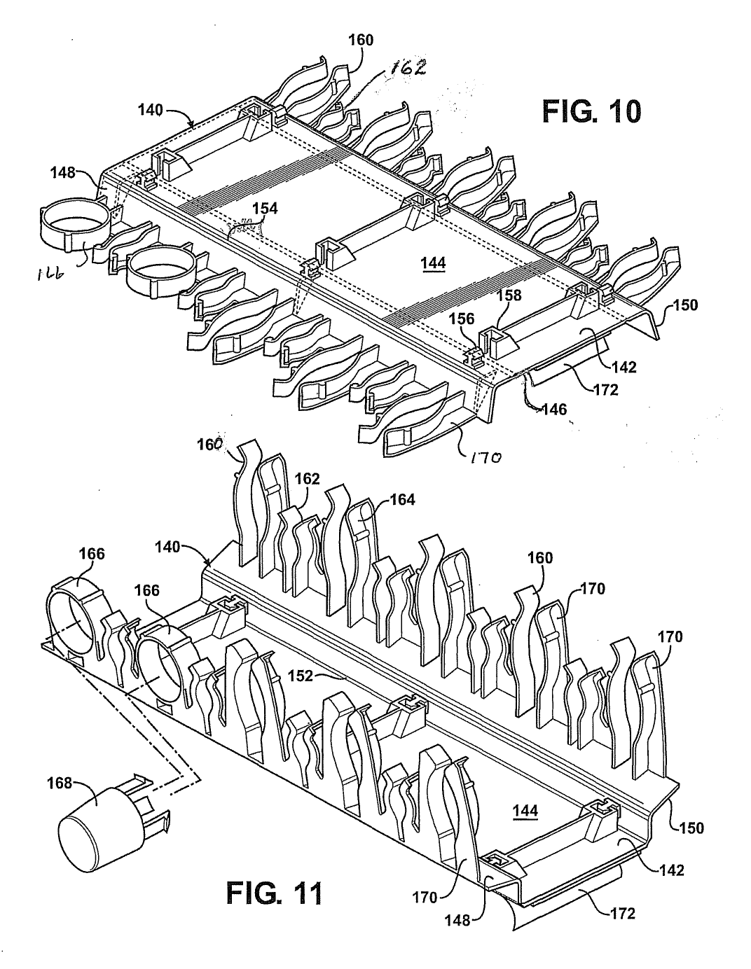

FIGS. 10 and 11 illustrate still another instrument holder 140 similar to holder 84 in that it comprises an elongate, generally flat plastic body 142 having generally planar top and bottom surfaces 144 and 146, respectively, and laterally opposite side wing structures 148, 150 attached to the body by living hinges 152, 154. The body 142 is molded substantially flat for ease of molding and packaging. In use, the wing structures are folded up and fastened by male spears 156 and female clips 158 similar to the fasteners 96, 98 of FIG. 5. In use, the wing structures 148, 150 carry laterally spaced and opposed sets of instrument receivers most of which are in the form of open-ended spring finger sets 160, 162, 164 of various sizes. In this embodiment, two of the receivers have spring fingers 164 on one side and an open loop 166 on the opposite side. In addition, a small plastic cup 168 may be detachably clipped to the loop 166. Alternatively the cup 168 may be formed integrally with the loop 166. The combination of the spring clip 164 on one side and the loop 166 and cup 168 on the other side provides a secure resting place for an instrument that might have a hot end; i.e., that hot end will fit into the cup to protect against accidental burns.

All of the receiver structures are molded with integral edge webs 170 for added structural strength.

The bottom of body 142 is generally planar and is preferably equipped with two-sided tape 172 so it may be adhered to a hose organizer or directly to a surgical drape, or to any other suitable surface in or near the site.

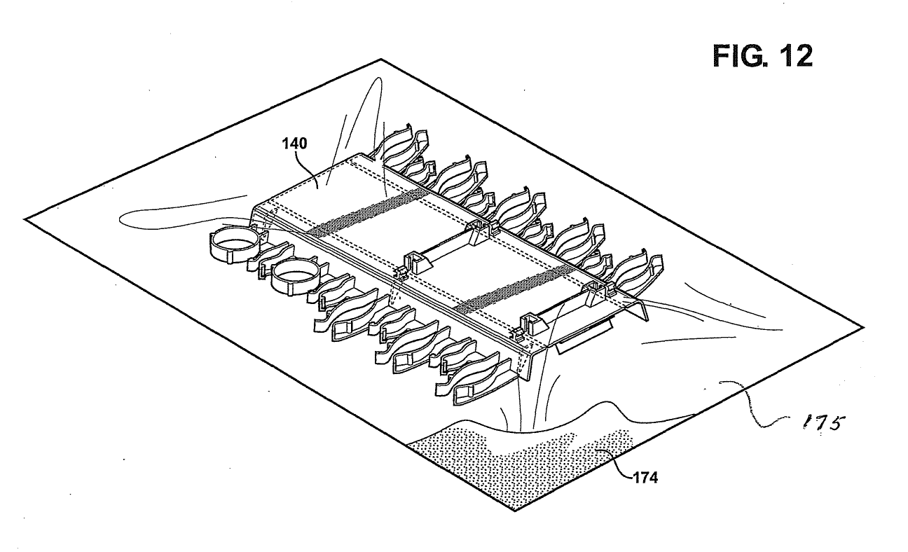

FIG. 12 shows a sterilized holder 140 in a sterile package comprising a layer 174 of gas-permeable material such as Tyvek and an attached clear layer 175 of plastic sheet.

The holder may be made of polypropylene in semi-transparent condition or other suitable material.

It will be apparent from the foregoing that uses of the organizer of the present invention are numerous and extend to dental work, podiatry and non-medical applications such as the organization of computer cables and the like.

What is claimed is:

1. An instrument holder comprising:
a body of substantially shape-retaining material having a top surface and a bottom surface; and
a plurality of parallel sets of upstanding instrument receivers mounted to and arranged along opposite edges of said body in upstanding relationship to said top surface to receive handheld elongate instruments therein.

2. An instrument holder as defined in claim 1 wherein at least one of said sets of receivers comprises first and second pairs of opposed, upstanding spring fingers, said first and second sets of upstanding opposed spring fingers being laterally spaced from one another across said body.

3. An instrument holder as defined in claim 2 wherein the first and second sets of spring fingers are about three to four inches apart.

4. An instrument holder as defined in claim 1 wherein at least one of said sets of receivers includes a closed loop of such diameter as to receive therein an elongate, handheld surgical instrument or a portion thereof and, opposing and spaced from said loop, a pair of spring fingers.

5. An instrument holder as defined in claim 1 wherein one of said sets of receivers includes a cup.

6. A holder for elongate objects comprising:
a body of substantially rigid material having a top surface and a substantially planar bottom and opposite edges; and
a plurality of receivers for elongate handheld objects arranged in opposed spaced-apart sets along said opposite edges.

7. A holder as defined in claim 6 wherein said receivers are of different sizes.

8. A holder as defined in claim 6 wherein at least one set comprises two pair of laterally spaced apart opposed spring fingers.

9. A holder as defined in claim 6 wherein at least one set comprises a closed loop on one edge for receiving a portion of a handheld surgical instrument therein and, aligned with said loop and laterally spaced therefrom, a pair of spring fingers.

10. A holder as defined in claim 6 wherein one of said receivers comprises a spring clip and, spaced from said clip, a cup.

11. A holder as defined in claim 6 wherein the material of construction is plastic.

12. A holder as defined in claim 6 wherein at least one receiver comprises a pair of open and opposed spring fingers upstanding from one edge of said body and a cup aligned with said spring fingers and upstanding from the opposite lateral edge to receive therein the active end of a surgical instrument such as a Bovie.

13. A holder as defined in claim 12 wherein the cup is detachably mounted to a closed loop which, in turn, is integral with said body.

14. A holder for handheld elongate surgical instruments comprising:
a generally planar molded plastic body having first and second parallel longitudinal wings attached to said body by integral hinges;
fastener means for securing said wings in a folded orientation relative to said body; and
a plurality of sets of instrument receivers arranged in first and second coacting portions along said wings.

15. A surgical instrument holder as defined in claim 14 wherein at least one of said receivers comprises on one of said edges, a pair of opposed spring clips.

16. A surgical instrument holder as defined in claim 14 wherein at least one of said receivers comprises along one edge a pair of opposed spring clips and, aligned therewith on the opposite edges of said body, a closed loop.

17. An instrument holder as defined in claim 14 wherein at least one of said receiver comprises a pair of opposed spring clip fingers upstanding from one of said wings and, opposite said fingers and aligned therewith on the other of said wings, a cup for receiving the active end of a surgical instrument such as a Bovie.

18. A surgical instrument holder as defined in claim 13 including webs molded integral with said receivers for adding structural strength thereto.

19. In combination, a surgical instrument holder as defined in claim 14 disposed within a closed sterilizable package comprising a sheet of gas permeable material.

20. A method of presenting handheld surgical instruments for use during a procedure comprising the steps of:
de-packaging a sterile instrument holder;
reconfiguring the holder from the packaged condition to a use condition;
attaching the holder to a support surface; and
placing surgical instruments on or in the holder in the reconfigured condition.
